(19) United States
(12) Patent Application Publication
(10) Pub. No.: US 2015/0065686 A1
(43) Pub. Date: Mar. 5, 2015

(54) METHOD FOR MAKING A FIBRE COMPRISING NATURAL FIBRE NANOPARTICLES

(71) Applicant: HERIOT WATT UNIVERSITY, Galashiels (GB)

(72) Inventor: George Stylios, Selkirkshire (GB)

(21) Appl. No.: 14/391,550

(22) PCT Filed: Apr. 2, 2013
(86) PCT No.: PCT/GB2013/000148

§ 371 Date: Oct. 9, 2014

(30) Foreign Application Priority Data
Apr. 2, 2012 (GB) 1205916.8

(51) Int. Cl. D01D 5/00; C07K 14/78; D01D 5/06

(52) U.S. Cl. CPC D01D 5/0046; D01D 5/06; C07K 14/78
USPC 530/357; 264/176.1; 264/466; 530/353

(57) ABSTRACT

A method for making natural fibre products comprising embrittling natural fibres and breaking the embrittled fibres into nanoparticles, forming a suspension of the fibre nanoparticles in a spinnable liquid, and spinning fibre from the suspension. The method may be used to make fibres having the dimensions and properties of cashmere.

This invention relates to the production of fibre.

Natural fibres are vegetable fibres such as cotton and flax or animal fibres such as wool and silk. Man-made fibres include cellulose fibres, rayon and viscose, obtained from plant cellulose by dissolving wood pulp and spinning fibre from the solution, and synthetic fibres such as polyethylene and nylon spun from molten polymer material.

Natural fibres have developed over millennia to be fit for purposes that correspond to the requirements of textiles, and are widely used in the manufacture of fabrics for garments and other products. Until man-made and fully synthetic fibres were developed, natural fibres were the only fibres available for such purposes. Nowadays, they are still very much in demand, and command premium prices, because their properties are generally speaking superior, particularly in terms of appearance and ‘handle’—a somewhat subjective term that means different things to different people, but is concerned with the way a fabric feels and behaves. People assess the handle of natural fibres to be distinct from, and usually preferable to the handle of man-made and synthetic materials.

Some natural fibres are more highly prized than others. Cashmere is the fine hair from a goat, and is a premium textile on account of its fineness of texture, light weight, strength and heat insulating properties. It is readily spun into yarns and woven or knitted into fabrics. The average annual yield of cashmere fibre from a goat is about 150 grammes. Except for the Angora goat, the coat is comprised of the fine hair and coarse fibre known as guard hair, which can account for some 80% of the weight of the fleece, and must be separated out by combing. While there are uses for the guard hair, such as in brushes or mats, most of it is simply thrown away. Cashmere is an expensive fibre.

Sheep are grown primarily for meat, but also produce substantial quantities of wool. While wool is a premium fibre for many end uses, men's suitings and sweaters being examples, considerably more wool, is produced than can be used for such products.

The present invention provides novel textile fibre materials and novel ways of making textile fibre materials that are essentially natural fibres but that can have enhanced properties such as are found in normally high-value fibres but at reduced cost.

The invention comprises a method for making natural fibre products comprising embrittling natural fibres and breaking the embrittled fibres into nanoparticles, forming a suspension of the fibre nanoparticles in a spinnable liquid, and spinning fibre from the suspension.

The fibres may be embrittled by freezing, as by being cooled to −40° C., and may then be milled or ground into nanoparticles in such manner as preserves their chemical identity and properties. The nanoparticles may have a maximum dimension of 30 microns or less, preferably 15 microns or less. The fibres may comprise guard hair separated during cashmere production.

The spinnable liquid may comprise fibroin, which may be recovered from cocoons of the silkworm (Bombyx mori).

Cocoons may be degummed by boiling one or more times in 0.5% (w/v) Na₂CO₃ solution in de-ionized water, after which they may be washed with de-ionized water. They may then be dried at room temperature.

The resulting dried silk may then be dissolved in a solution of CaCl₂:water:ethanol, 1:8:2 molar ratio at 80° C. for two hours, and this silk solution treated to remove salts, for example by dialysis in cellulose tubing against de-ionised water. Dialysis may be continued for 4-5 days.

The dialysed solution may be filtered and air-dried at room temperature. This may take 2-3 days. The dried silk may then be dissolved in formic acid at 15% (w/v) and stirred in an ultrasonic shaker. This may be continued for 2-3 days.

The nanoparticles of hair may then be added to this solution and stirred again to form a well-mixed suspension. This may take 2-3 days in an ultrasonic shaker

The suspension may then be electrospun. It may be electrospun from a 22-gauge needle with a 6000V potential difference to a collector at a distance of 15 cm, at a feed rate of 0.1 ml/hr.

If the collector is a wire card with earthed wires, the fibres may be collected in orderly fashion and undergo the usual procedures of carding, twisting and plying to make yarns, cables, ropes and other elongate structures that can be knitted or woven into fabrics for garments and other end uses.

The suspension may also be wet spun, and may be wet spun, for example, into a methanol solution.

The invention also comprises a spun fibre comprising nanoparticles of natural fibre. The natural fibre nanoparticles may be wool particles, and may be of cashmere. The nanoparticles may be comprised in a fibroin fibre.

The fibroin may be spun without the inclusion of the nanoparticles, but there needs only to be enough fibroin to constitute a matrix to hold the nanoparticles, and so the nanoparticles may be present in an amount from 0 to 80% (w/w).

The spun fibre may be collected as a non-woven fabric or may, particularly when electrospinning is used, be collected on a card wire and further processed by carding and other textile operations to make spun yarn which can be used to make knitted or woven fabrics, and the invention includes roving, yarns, cables, ropes and other elongate structures comprising the fibre and woven and knitted fabrics made therefrom.

Natural fibre products and methods for making them according to the invention will now be described with reference to the accompanying drawings, in which:

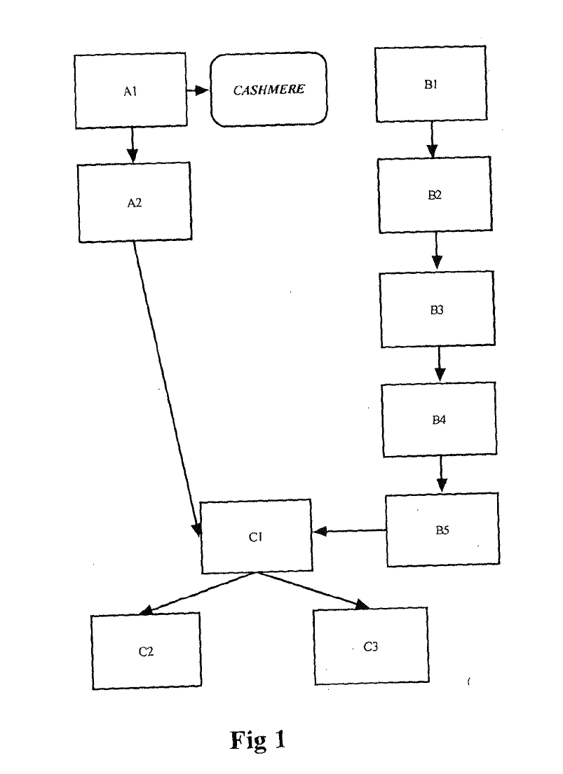

FIG. 1 is a block diagram of processes for making a cashmere fibre; and

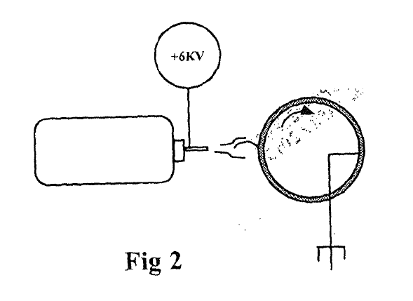

FIG. 2 is a diagram of an electrospinning arrangement.

The drawings illustrate a method for making natural fibre products comprising embrittling by embrittling natural fibres and breaking the embrittled fibres into nanoparticles, forming a suspension of the fibre nanoparticles in a spinnable liquid and spinning fibre from the suspension.

Guard hair from a goat is separated from the finer cashmere fibres, step A1, FIG. 1, the cashmere fibres being sent for processing in the normal way.

In step A2, the guard fibres embrittled by freezing, by being cooled to −40° C. and ground or milled into nanoparticles in such manner as preserves their chemical identity and properties. The nanoparticles have a maximum dimension of 30 microns or less, preferably 15 microns or less.

The spinnable liquid comprises fibroin, recovered from cocoons of the silkworm (Bombyx mori).

Cocoons are degummed, step B1, FIG. 1, by boiling one or more times in 0.5% (w/v) Na2CO3 solution in de-ionized water, after which they are washed with de-ionized water. They are then dried at room temperature.

The resulting dried silk is then dissolved, step B2 in a solution of CaCl2: water:ethanol, 1:8:2 molar ratio at 80° C. for two hours, and this silk solution treated to remove salts, by dialysis in cellulose tubing against de-ionised water, step B3. Dialysis is continued for 4-5 days.

The dialysed solution is filtered, step B4, and air-dried at room temperature. This may take 2-3 days. The dried silk is then, step B5, dissolved in formic acid at 15% (w/v) and stirred in an ultrasonic shaker. This is continued for 2-3 days.

The nanoparticles of hair are added to this solution, step C1, and stirred again to form a well-mixed suspension. This takes 2-3 days in an ultrasonic shaker

The suspension is then electrospun, step C2.

The electrospinning arrangement is shown in FIG. 2. A container 21 for the suspension has a nozzle 22 comprising a 22-gauge needle 23, FIG. 2. An electrostatic arrangement 24 maintains with a 6000V potential difference to a collector 25 which comprises a carding engine, of which only the swift is shown. The collector 25 is at a distance of 15 cm fro the needle 23, and the suspension is directed towards the collector 25 at a feed rate of 0.1 ml/hr.

The collector 25 is a wire card with earthed wires, on which the fibres are collected in orderly fashion and undergo the usual procedures of carding, twisting and plying to make yarns, cables, ropes and other elongate structures that can be knitted or woven into fabrics for garments and other end uses.

Alternatively, the suspension may go to a conventional wet spinning process, step C3, FIG. 1, into a methanol solution.

1. A method for making natural fibre products comprising embrittling natural fibres and breaking the embrittled fibres into fibre nanoparticles, forming a suspension of the fibre nanoparticles in a spinnable liquid, and spinning fibre from the suspension.

2. A method according to claim 1, wherein the fibres are embrittled by freezing and then ground or milled into the fibre nanoparticles in such manner as preserves their chemical identity and properties.

3. A method according to claim 2, wherein the fibre nanoparticles have a maximum dimension of 30 microns or less.

4. A method according to claim 1, wherein the fibres comprise guard hair separated during cashmere production.

5. A method according to claim 1, wherein the spinnable liquid comprises fibroin.

6. A method according to claim 5, wherein the fibroin is recovered from cocoons of the silkworm (Bombyx mori).

7. A method according to claim 6, wherein cocoons are degummed by boiling one or more times in 0.5% (w/v) Na₂CO₃ solution in de-ionized water.

8. A method according to claim 7, wherein after degumming the cocoons are washed with de-ionized water and dried at room temperature.

9. A method according to claim 6, wherein the fibroin is dissolved in a solution of CaCl₂:water:ethanol, 1:8:2 molar ratio at 80° C. for two hours, and then treated to remove salts.

10. A method according to claim 9, wherein the salts are removed from the solution by dialysis.

11. A method according to claim 10, wherein the dialysis is carried out in cellulose tubing against de-ionised water.

12. A method according to claim 10, wherein the dialysis is continued for 4-5 days.

13. A method according to claim 10, wherein after the dialysis the solution is filtered and air-dried at room temperature.

14. A method according to claim 6, wherein the fibroin is dissolved in formic acid at 15% (w/v) and stirred in an ultrasonic shaker and the fibre nanoparticles are added thereto to form a mixed suspension.

15. A method according to claim 1, wherein he suspension is electrospun.

16. A method according to claim 15, wherein the suspension is electrospun from a 22-gauge needle with a 6000V potential difference to a collector at a distance of 15 cm, at a feed rate of 0.1 ml/hr.

17. A method according to claim 15, wherein the collector is a wire card with earthed wires.

18. A method according to claim 1, wherein the suspension is wet spun.

19. A method according to claim 18, wherein the suspension is spun into a methanol solution.

20. A spun fibre comprising nanoparticles of natural fibre.

21. A spun fibre according to claim 20, wherein the nanoparticles of natural fibre are wool particles.

22. A spun fibre according to claim 23, wherein the wool particles comprises cashmere.

23. A sun fibre according to claim 20, wherein the nanoparticles of natural fibre are comprised in a fibroin fibre.
